(19) United States
(12) Patent Application Publication
(10) Pub. No.: US 2008/0300665 A1
(43) Pub. Date: Dec. 4, 2008

(54) MEDICAL IMPLANT, IN PARTICULAR STENT

(75) Inventors: Daniel Lootz, Rostock (DE); Alexander Winter, Rostock (DE)

Correspondence Address: POWELL GOLDSTEIN LLP, ONE ATLANTIC CENTER FOURTEENTH FLOOR, 1201 WEST PEACHTREE STREET NW, ATLANTA, GA, 30309-3488, US

(73) Assignee: BIOTRONIK VI PATENT AG, Baar (CH)

(21) Appl. No.: 12/131,174

(22) Filed: Jun. 2, 2008

(30) Foreign Application Priority Data
Jun. 2, 2007 (DE) 10 2007 025 921.4

(51) Int. Cl. A61F 2/86

(52) U.S. Cl. 623/1.2

(57) ABSTRACT

A medical implantable stent comprising a main structure (1) composed of deformable struts (2), which are preferably machined from a tubular blank (3) by laser cutting. A fine structure (4) made of a wire-like or thread-like material attached to the struts (2) of the main structure (1).

### PRIORITY CLAIM

This patent application claims priority to German Patent Application No. 10 2007 025 921.4, filed Jun. 2, 2007, the disclosure of which is incorporated herein by reference in its entirety.

### FIELD

The present disclosure relates to a medical implant and, in particular, a stent having a main structure composed of deformable struts, which is preferably machined from a tubular blank by laser cutting. Alternative production methods, for example, laser sinter rapid prototyping, are also conceivable.

### BACKGROUND

Medically implantable stents are known in various embodiments, the typically laser-cut struts making use of shaped elements, such as ring segments, spirals, axial connectors, and the like, in their main structure. The connection between these various strut structures is fundamentally a material bond because of the production method. This means that the shape changes of the stent required for medical use upon application from a catheter, for example, are to be implemented by a deformation of the stent material itself. Deformations of this type may occur elastically or also plastically, the limits for such a deformation being materially dependent and thus possibly also very strongly restricted. Furthermore, material bonds have the property of transmitting forces both in the compression and also in the traction direction. For relatively small deformations in the elastic range, the transmission properties are direction independent in this case. A very flexible connection in the compression direction also automatically has a high flexibility in the traction direction for small deformations. This is also true for rigid connections. For example, the mechanical properties of stents constructed according, for example, to U.S. Pat. No. 5,102,417 may also only be designed to a limited extent as a function of the load direction due to the material bond of the strut structures. This may have a disadvantageous effect on the properties of a stent under bending load, for example. A very soft deformation behavior on the compression side, but a slight deformation on the traction side may be desirable here to maintain a tubular external contour which is simultaneously adapted as well as possible to the blood vessel with overall low bending stiffness.

Braided stents represent a further construction of medical implants, in particular stents. Braided stents are known, for example, from U.S. Pat. No. 7,001,425 or U.S. Pat. No. 6,342,068. The braided structure without material bonds allows large relative movements between crossing wires in these implants, which provides the stents with comparatively high flexibility. The absorbable radial forces are comparatively low for this stent type. Fundamentally, a pronounced length change occurs upon stent expansion or compression, which is generally undesirable. Furthermore, bending of the stent is connected to a relatively strong reduction of the cross-sectional area which may have flow through it.

Retractable stents represent a special case. They are distinguished in that, after a partial release from the insertion system, the stents may be retracted back therein, to perform repositioning in the vessel, for example. A stent of this type is known, for example, from U.S. Pat. No. 7,037,331. To ensure the retractability, the structure of such a stent must be completely cross-linked so that no free ends exist within the structure which could get caught upon retraction into the release system. This circumstance makes stents constructed in this manner comparatively resistant to bending which makes good adaptation to strongly contorted vessels more difficult.

An open-celled stent, as described in U.S. Pat. No. 7,037,330, does have lower bending stiffness but may not be retracted because of the free ends within the structure and tends to deviate strongly from the desired tubular external contour under bending strain.

### SUMMARY

The present disclosure describes several exemplary embodiments of the present invention.

One aspect of the present disclosure provides a medical implant, in particular a stent, comprising a main structure composed of materially bonded deformable struts, machined from a tubular blank by laser cutting, further comprising a fine structure, attached to the struts of the main structure, made of a wire-like or thread-like material.

One aspect of the present disclosure provides a medical implant and, in particular, a stent having a main structure composed of deformable struts in such a manner that while maintaining reasonable absorbable radial forces, a high flexibility is ensured in the bending direction with optimum surface coverage and cross-linking density, without losing the tubular external contour. Another aspect of the present disclosure provides retractable stents having greatly improved bending flexibility.

One feature of laser-cut implants, which allow deformations only through comparatively high elastic or plastic strain of the material, may be combined with the features of braided and/or woven implants by this combination. The condition for a retractable stent concurrently having low bending stiffness is thus also provided.

According to at least one exemplary embodiment of the present disclosure, the fine structure has at least one wire or thread which is connected to the main structure on at least one attachment point loosely, in a formfitting manner, materially bonded, by gluing or by a fixing thread. Because of the selection capability among various attachments between fine structure and main structure, the stent according to this embodiment may be adapted especially well to the desired intended purpose. Further exemplary embodiments relate to constructive details for the connection of wire or thread to the main structure, in whose context reference is made to the description of the exemplary embodiments to avoid unnecessary repetitions.

Further exemplary embodiments relate to the assignment of the fine structure to a main structure made of helical peripheral meandering struts. Reference is also made here to the corresponding passages of the description of the exemplary embodiments to avoid unnecessary repetitions.

According to a further exemplary embodiment, the fine structure of the wire or thread forming the implant has a microstructure itself to expand its functionality. Thus, wire or thread may be composed of two or more materials so that multicomponent layered or composite systems may be produced relatively simply, for example. Structures in the form of wires or threads have the specific advantage that they may be produced as semifinished products in endless manufacturing and subsequently installed in the appropriate configuration in the implant. Thus, for example, a combination of a stable biocompatible material with a degradable polymer which, as a coating charged with active ingredient, acts as a medication depot, is to be mentioned. If the fine structure is a thread, it may in turn be impregnated with polymer which provides the advantage of better ability to be incorporated than the application of an active ingredient combination on the surface of a metallic structure.

Further material combinations may relate, for example, to the coating of the wire or thread surface with silicon carbide to improve the biocompatibility or the incorporation of an x-ray-opaque material in the wire or thread. Thus, for example, a wire may be provided with a coating or a core made of x-ray-opaque material.

According to a further exemplary embodiment of the present disclosure, the wire or thread may be implemented as a traction element for retracting the implant into a contracted state. In this context, it is advantageous that no separate constructive element is required for the retraction of the implant because the fine structure in the form of the wire or thread, which is part of the implant, merely has to be continued from the implant and may be pulled out as an attack element to retract the implant.

### BRIEF DESCRIPTION OF THE DRAWINGS

Various aspects of the present disclosure are described hereinbelow with reference to the accompanying figures in which like reference number refer to like parts.

FIG. 1 shows a schematic view of a stent having fine structures in one exemplary embodiment;

FIG. 2 shows a schematic view of a stent having fine structures in a second exemplary embodiment;

FIG. 3 shows a schematic view of a stent having fine structures in a third exemplary embodiment;

FIG. 4 shows a schematic view of a stent having fine structures in a fourth exemplary embodiment;

FIG. 5 shows a schematic view of a stent having fine structures in a fifth exemplary embodiment;

FIG. 6 shows a view of attachment points between the fine and coarse structures of a stent in one exemplary embodiment;

FIG. 7 shows a view of attachment points between the fine and coarse structures of a stent in an exemplary embodiment;

FIG. 8 shows a view of attachment points between the fine and coarse structures of a stent in an exemplary embodiment;

FIG. 9 shows a view of attachment points between the fine and coarse structures of a stent in an exemplary embodiment;

FIG. 10 shows a view of attachment points between the fine and coarse structures of a stent in an exemplary embodiment;

FIG. 11 shows a view of attachment points between the fine and coarse structures of a stent in an exemplary embodiment;

FIG. 12 shows a view of attachment points between the fine and coarse structures of a stent in an exemplary embodiment;

FIG. 13 shows a view of attachment points between the fine and coarse structures of a stent in an exemplary embodiment;

FIG. 14 shows a view of attachment points between the fine and coarse structures of a stent in an exemplary embodiment;

FIG. 15 shows a sectional perspective illustration of threads or wires of the fine structure of the stent in one exemplary embodiment;

FIG. 16 shows a sectional perspective illustration of threads or wires of the fine structure of the stent in an exemplary embodiment;

FIG. 17 shows a sectional perspective illustration of threads or wires of the fine structure of the stent in an exemplary embodiment;

FIG. 18 shows a sectional perspective illustration of threads or wires of the fine structure of the stent in an exemplary embodiment;

FIG. 19 shows a sectional perspective illustration of threads or wires of the fine structure of the stent in an exemplary embodiment;

FIG. 20 shows a sectional perspective illustration of threads or wires of the fine structure of the stent in an exemplary embodiment;

FIG. 21 shows a sectional perspective illustration of threads or wires of the fine structure of the stent in an exemplary embodiment;

FIG. 22 shows a sectional perspective illustration of threads or wires of the fine structure of the stent in an exemplary embodiment; and

FIG. 23 shows a sectional perspective illustration of threads or wires of the fine structure of the stent in an exemplary embodiment.

### DETAILED DESCRIPTION

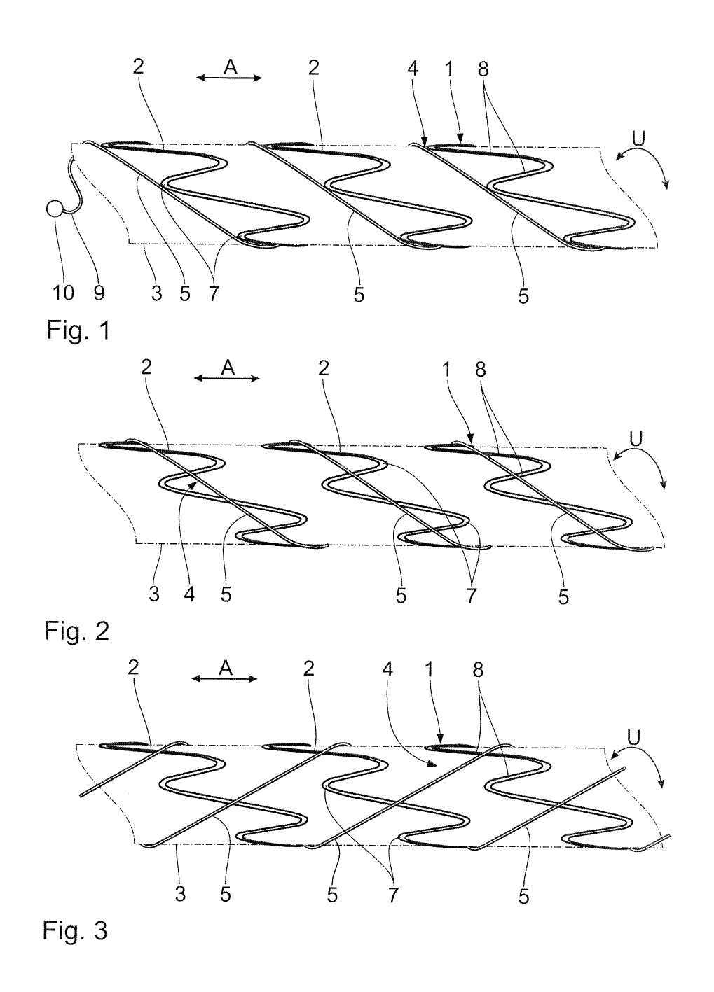

FIGS. 1-5 show a stent which has a main structure 1 composed of meandering struts 2 which have been machined by laser cutting from a blank 3 indicated in FIGS. 1-5. The main structure 1 circumscribes a cylindrical space, the struts 2 running helically in the peripheral direction U. The struts 2 are radially expandable in a known way, for example, by a catheter balloon, to apply the stent in a coronary vessel of the heart, for example, because of their meandering course.

The main structure 1 has a fine structure, identified as a whole by part number 4, superimposed, which is applied in various configurations in FIGS. 1-5. Fundamentally, the fine structure 4 comprises a wire/thread 5 which is attached to the main structure 1 in a manner to be explained in greater detail.

In the exemplary embodiment shown in FIG. 1, the wire/thread 5 runs helically around the stent and along each of the zeniths 7 of the meandering curves 8. The wire/thread 5 is extended beyond the struts 2 at one end of the stent, and a coupling element 10 is attached to its protruding end 9 on which a retraction mechanism for a radial contraction of the stent may engage.

In the exemplary embodiment shown in FIG. 2, the wire/thread 5 runs centrally between the zeniths 7, also helically around the stent. In the exemplary embodiments shown in FIGS. 1 and 2, the wire/thread 5 thus runs parallel to the helical turns of the strut 2.

FIG. 3 shows an exemplary embodiment in which the turns of the struts 2 and the also helical peripheral wire/thread 5 run in opposite directions so that struts 2 and wire/thread 5 intersect like a honeycomb.

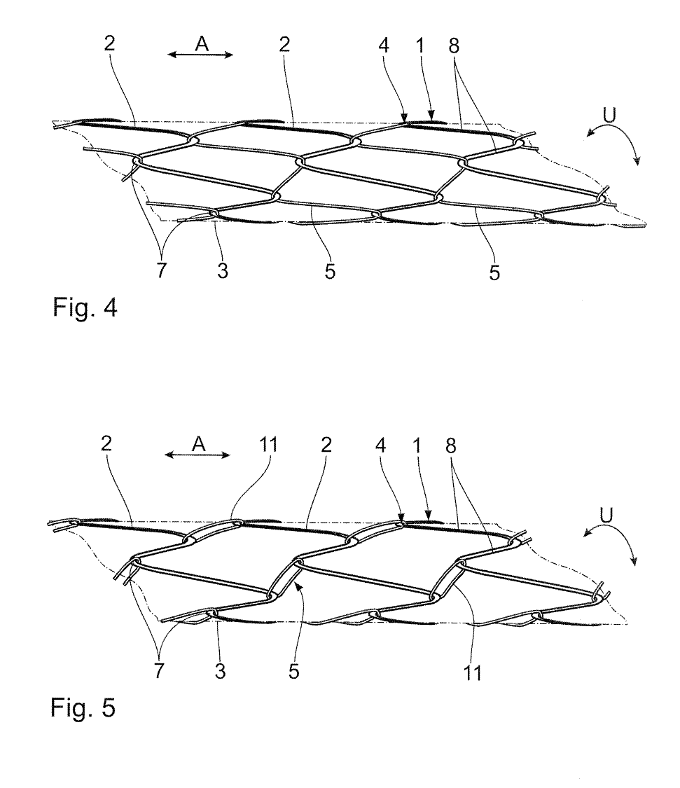

In a further exemplary embodiment, as shown in FIG. 4, a thread/wire 5 is led back and forth in a zigzag between the zeniths 7 of two adjacent turns of the struts 2 so that the adjacent coils are woven with one another.

Finally, FIG. 5 shows an exemplary embodiment in which a thread/wire 5 is wound around as a fixing ring 11 between the zeniths 7 of two adjacent turns of the struts 2 in each case.

It is also to be noted that in all exemplary embodiments according to FIGS. 1-5, axial connectors (not shown) may be provided in the axial direction A between two struts running adjacent to one another around the circumference, if this is necessary for reasons of special structural stability of the stent.

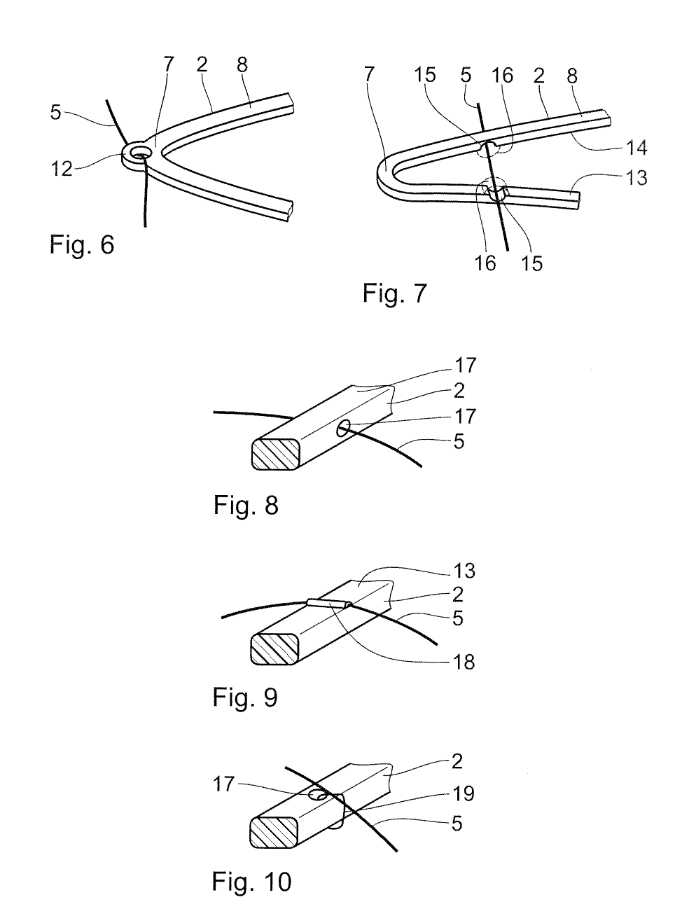

The various possibilities for how a wire/thread 5 may be attached to a strut 2 of the main structure 1 will now be explained on the basis of FIGS. 6-14. Thus, FIG. 6 shows an eye 12 on the zenith 7 of a meandering curve 8 of the struts 2 through which the wire/thread 5 runs loosely.

In FIG. 7, the wire/thread 5 runs approximately centrally over two adjacent sections of a meandering curve 8 in a depression 15 molded in opposite directions on the top and bottom sides 13, 14. This depression may be implemented as a notch which is semi-cylindrical, triangular, or rectangular in cross-section. As indicated by dashed lines in FIG. 7, the wire/thread 5 may also be fixed on the strut 2 by a glued bond 16, for example, so that a relative movement is not possible between strut 2 and wire/thread 5.

FIG. 8 shows the guiding of a wire/thread 5 through a hole 17 in the strut 2.

The welding/soldering/gluing shown in FIG. 9, or another type of joining technique 18 on the top side 13 of the strut 2, ensures a material bond of wire/thread 5 to strut 2.

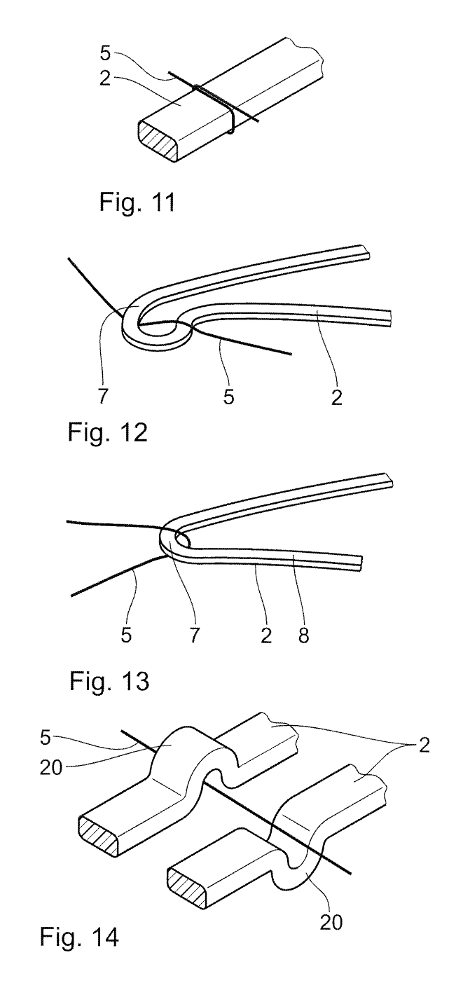

In FIG. 10, the wire/thread 5 is laid on the strut 2 in the area of a hole 17 through which a fixing thread/fixing wire 19 is then drawn and the wire/thread 5 is thus guided on the strut 2. The wire/thread 5 is wound around the strut 2 in FIG. 11

In the exemplary embodiment shown in FIG. 12, a special eye-like shaped area of the strut 2 in the area of its zenith 7 ensures good guiding of the wire/thread 5.

FIG. 13 shows the course of the wire/thread 5 applied in a zigzag shown in FIG. 4 in the area of the zenith 7 of a meandering curve in detail.

In the exemplary embodiment from FIG. 14, the struts 2 each have shaped areas 20 in which the thread 6 is laid.

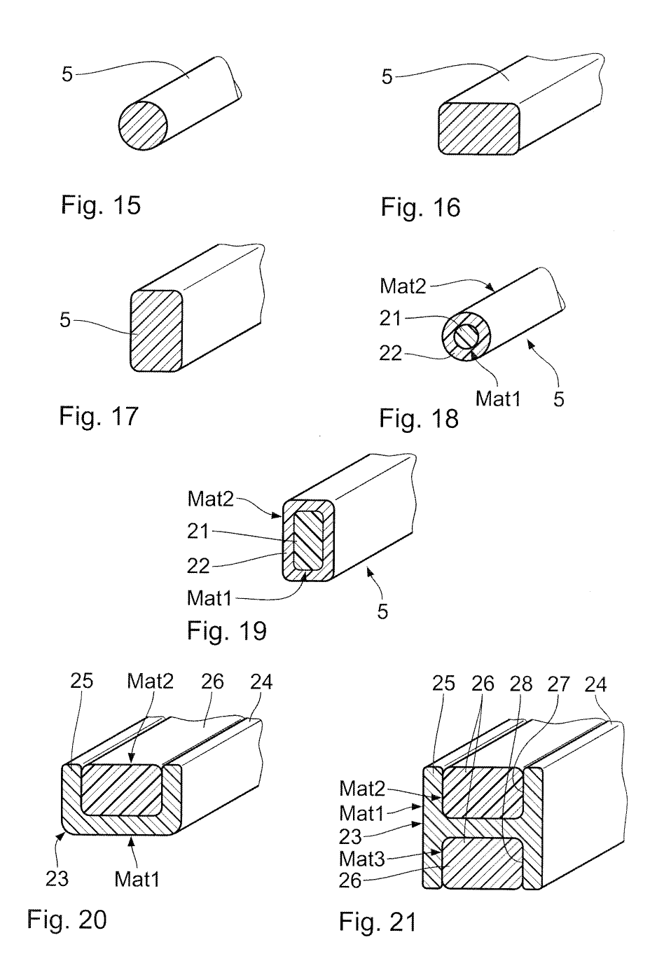

Various exemplary embodiments of the wire/thread 5 are illustrated in regard to a microstructure integrated therein in FIGS. 15-23.

FIG. 15 shows the simplest exemplary embodiment, in which a thread 5 comprises a biologically degradable polymer material, for example. A resistant or degradable wire may also be used according to the present disclosure.

FIGS. 16 and 17 show wires 5 which have a flat-rectangular cross-section. Wires 5 of this type are to be effectively provided with a smooth surface and finish, for example, by pickling or electropolishing, because of the planar surface. Because of their direction-dependent bending stiffness, the wires 5 from FIGS. 16 and 17 may be attached to the main structure 1 in a specific installation direction to influence the bending behavior of the entire stent.

FIGS. 18-21 and 23 show exemplary embodiments in which two different materials may be combined with one another. Thus, in the exemplary embodiment shown in FIG. 18, a round wire 5 is provided with a core 21 made of medical steel (or Nitinol) which is provided with a sheath 22 made of a biocompatible or biologically degradable polymer. FIG. 19 shows a corresponding construction for a wire 5 having a flat-rectangular cross-section.

In the exemplary embodiment shown in FIG. 20, a supporting profile 23 in U-shape is provided between whose legs 24, 25 a second material may be integrated, for example, a medication depot 26 in the form of active ingredients embedded in a degradable polymer.

In the exemplary embodiment shown in FIG. 21, the supporting profile 23 is H-shaped so that two receptacle grooves 27, 28 may be formed, which may in turn be filled with corresponding further materials.

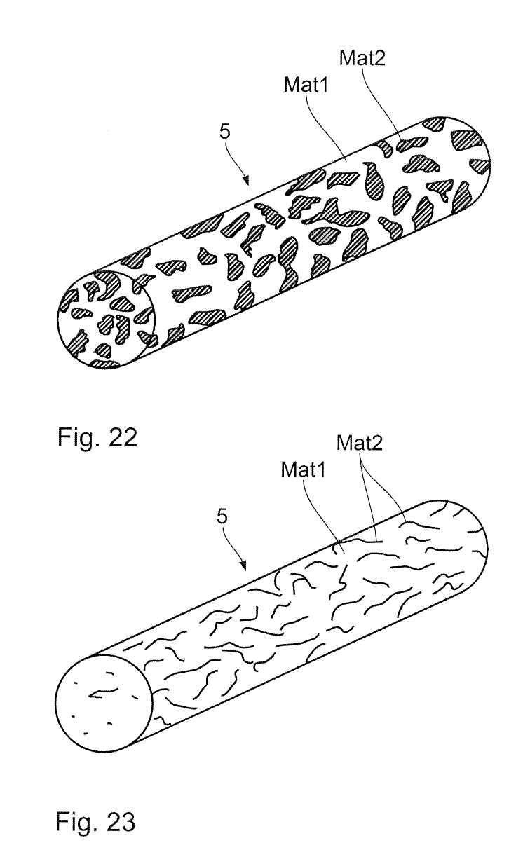

FIG. 22 shows a porous wire/thread 5 which has open-celled or close-celled cavities which may be unfilled or may contain a further material (Mat2) for functional expansion. FIG. 23 shows a fiber-reinforced or particle-reinforced thread-shaped or wire-shaped structure 5.

Moreover, material combinations of three materials (Mat1, Mat2, Mat3) may be inferred from the following Table 1, as they may be implemented in the exemplary embodiments from FIGS. 18-23.

TABLE 1

| | Mat 1 | Mat 2 | Mat 3 |
|---|---|---|---|
| FIGS. 18, 19: | Biocompatible metal, | Platinum, tantalum, . . . - | |
| | e.g., Nitinol | x-ray-opaque material | |
| FIG. 20: | biocompatible metal | Polymer | |
| | biocompatible metal | Polymer | |
| | biocompatible metal | Platinum, tantalum, . . . - | |
| | | x-ray-opaque material | |
| FIG. 21: | biocompatible metal | Polymer - active | Polymer - active |
| | | ingredient/active | ingredient/active |
| | | ingredient combination 1 | ingredient |
| | | | combination 1 |
| | biocompatible metal | Polymer - active | Polymer - active |
| | | ingredient/active | ingredient/active |
| | | ingredient combination 1 | ingredient |
| | | | combination 2 |
| | biocompatible metal | Polymer - active | Platinum |
| | | ingredient/active | |
| | | ingredient combination 1 | |
| | biocompatible metal | Platinum, tantalum, . . . - x- | Polymer |
| | | ray-opaque material | |
| FIG. 22 | biocompatible metal | Polymer - active | |
| | | ingredient/active | |
| | | ingredient combination | |
| | biocompatible polymer | Polymer - active | |
| | | ingredient/active | |
| | | ingredient combination | |
| | biocompatible metal | | |
| | biocompatible polymer | | |
| | biocompatible polymer | Active ingredient/active | |
| | | ingredient combination | |
| FIG. 23 | biocompatible metal 1 | biocompatible metal 2 | |
| | biocompatible polymer 1 | biocompatible polymer 2 | |
| | biocompatible metal | Reinforcing fiber | |
| | biocompatible polymer | Reinforcing fiber | |

(“Metal” in Table 1 comprises metals and metal alloys.)

The alloy “Nitinol” is a superelastic structural material. Platinum is used to improve the x-ray visibility of the stent. The cited polymers may be implemented as both degradable and also having long-term stability. They may, as already explained above on the basis of the drawings, be used as an active ingredient carrier for so-called “drug-eluting applications”.

A non-superelastic material may also be used for the structural material (Mat1), for example, chromium-nickel and cobalt-chromium alloys, tantalum, titanium, niobium, and their alloys.

All patents, patent applications and publications referred to herein are incorporated by reference in their entirety.

What is claimed is:

1. A medical implant, in particular a stent, comprising:
a) a main structure comprising materially bonded deformable struts, machined from a tubular blank by laser cutting,
b) a fine structure, attached to the struts of the main structure, made of a wire-like or thread-like material.

2. The implant of claim 1, wherein the fine structure has at least one wire or thread connected to the main structure on at least one attachment point loosely, in a formfitting manner, materially bonded by either gluing or by a fixing thread.

3. The implant of claim 2, wherein the wire or thread is guided so that the wire or thread is displaceable in at least one receptacle on the main structure.

4. The implant of claim 3, wherein the receptacle is a notch-shaped depression, hole, or eye on a particular strut of the main structure.

5. The implant of claim 1, wherein the main structure is formed by helical peripheral, meandering struts, at least one wire or thread of the fine structure being attached in the area of either the zeniths of the meandering curves or centrally thereon.

6. The implant of claim 1, wherein the main structure is formed by helical peripheral, meandering struts, the at least one wire or thread of the fine structure being attached to the main structure parallel to the turns of the struts in an opposing helical configuration running between two adjacent turns of the struts either in a zigzag, or as fixing rings connecting two turns of struts to one another at adjacent zeniths.

7. The implant of claim 1, wherein the wire or thread has a microstructure to expand its functionality.

8. The implant of claim 7, wherein the wire or thread comprising two or more materials, which are selected from the group consisting of stable or degradable, biocompatible metal, stable or degradable polymer with or without medication depot, silicon as a coating material, x-ray-opaque material as a coating or core material.

9. The implant of claim 1, wherein the wire or thread is implemented as a traction element for retracting the implant into a contracted state
